(19) United States
(12) Patent Application Publication
(10) Pub. No.: US 2012/0048265 A1
(43) Pub. Date: Mar. 1, 2012

(54) AEROSOL DRUG DELIVERY APPARATUS AND METHOD

(75) Inventor: Gerard Smaldone, Stony Brook, NY (US)

(73) Assignee: KONINKLIJKE PHILIPS ELECTRONICS N.V., EINDHOVEN (NL)

(21) Appl. No.: 13/266,509

(22) PCT Filed: May 10, 2010
(86) PCT No.: PCT/IB2010/052060

§ 371 Date: Oct. 27, 2011

Related U.S. Application Data
(60) Provisional application No. 61/216,022, filed on May 11, 2009.

(51) Int. Cl. A61M 11/00

(52) U.S. Cl. 128/200.15

(57) ABSTRACT

A drug delivery apparatus (5, 5′, 5″, 5′″) for delivering an aerosol including a drug to a patient includes an aerosol generator (45, 50, 55, 60) for generating the aerosol from a supply of the drug, and a mouthpiece component (30, 30′, 30″, 30′″) structured to be inserted into the mouth of the patient. The mouthpiece component is operatively coupled to the aerosol generator for receiving the aerosol and delivering the aerosol to the patient. The mouthpiece component has a ventral surface (85, 85′, 85″) structured to face a tongue (100) of the patient when the mouthpiece component is inserted into the mouth of the patient and may include a tongue apex positioning element (90, 90′, 90′″, 105) provided on the ventral surface. The ventral surface, and if present, the tongue apex positioning element is/are structured to engage the apex (95) of the tongue of the patient during use of the drug delivery apparatus to properly position the tongue.

This application claims priority under 35 U.S.C. §119(e) from U.S. Provisional Application No. 61/216,022, entitled “Mouthpiece with Tongue Guide”, filed on May 11, 2009, the disclosure of which is incorporated herein by reference.

The present invention relates to the administration by a subject, per os, of aerosolized particles to the lung. More particularly, the invention relates to a mouthpiece that guides a subject to acquire and maintain, during administration, a tongue position that reduces deposition of the aerosolized particles in the oropharynx, without restricting the free passage air.

Inhalers, nebulizers, atomizers and other devices that create colloidal suspensions of dry or “wet” particles in air (i.e., “aerosols”) are commonplace. The device may provide the energy required for aerosolization, or the user of the device may do so, essentially by applying suction generated by inhaling. Subjects may introduce such aerosols into the nose or mouth or inhale the aerosols into the trachea and the lungs via the nose or mouth. Aerosols generated for inhalation typically comprise drugs targeted for either a local or a systemic site of action. In general, such drugs are innocuous to oropharyngeal tissues and, if not inhaled, they can be ingested with impunity. To the extent such drugs are also relatively inexpensive, their incidental deposition in the oropharynx is of little consequence. However, the list of expensive to very expensive therapeutic agents for which aerosol administration is acceptable, indicated, preferred or even essential is growing. A number of these agents, moreover, may be toxic outside of a narrow therapeutic index, such that they require special attention to dosage for safety reasons.

At the same time, it is preferable to avoid extreme or unpleasant measures such as endotracheal intubation, tongue depressors or other devices, e.g., metal or plastic conduits to eliminate orypharyngeal tissues as deposition sites. It is also advantageous to avoid devices that compromise or complicate the flow of inhaled air.

What is needed is a non-obstructive device that effectively assists subjects to voluntarily create a clear or at least reproducible inhalation flow-path through the oral cavity for the aerosol as it is being inhaled.

In its various embodiments, the invention addresses the problems of wasting drugs and introducing variability in dosage due to aerosolized particles depositing on the tongue during inhalation of an aerosol by a subject. Thus, in one embodiment, the invention provides a mouthpiece defining a breathing passage extending therethrough, the mouthpiece comprising a tongue guide. In one embodiment, an aerosol impelled by an inhalation pressure gradient flows from an aerosol-generating source, through the breathing passage and into the oral cavity along an inhalation airflow-path disposed around an inhalation airflow-path axis extending from the breathing passage to the trachea. In a preferred embodiment, the tongue guide is disposed on the mouthpiece such that (i) it does not interfere with the flow of air or aerosol along the inhalation airflow-path and (ii) the apex of the tongue fits underneath the tongue guide. In one embodiment, the tongue guide forms a roof of a tongue recess. In one embodiment, the tongue recess is sized to accept a portion of a tongue of a subject using the mouthpiece. In one embodiment, the tongue recess covers an apex of the tongue in the recess. In one embodiment, the roof of the tongue recess covers a dorsal surface of the tongue from the apex posteriorly about 3 cm, preferably less than 2 cm and most preferably less than 1 cm. In one embodiment, the roof does not cover a vallate papilla.

In some embodiments, the mouthpiece further comprises a nebulizer. In one embodiment, the nebulizer is disposed in proximity to the mouthpiece such that, when the nebulizer emits aerosol particles, the particles flow into the breathing passage as a subject inhales. In one embodiment, the nebulizer emits the aerosol particles directly into the inhalation airflow-path. In preferred embodiments, the tongue guide is configured so as not to interfere with the flow of air or aerosolized particles through the breathing passage, but to provide a surface such that a subject can position the subject's tongue underneath the inhalation airflow-path.

In a preferred embodiment, the inhalation airflow-path extends through the breathing passage, over the tongue into the trachea of the subject.

In one embodiment, the invention provides a method of controlling the flow of aerosolized particles inhaled by a subject, the method comprising: a) providing a device comprising a nebulizer, a mouthpiece, and a tongue guide, wherein the mouthpiece comprises a breathing passage extending therethrough, wherein the nebulizer is disposed in proximity to the mouthpiece such that, when the nebulizer emits aerosol particles, the particles flow into an inhalation airflow-path as a subject inhales, wherein the tongue guide extends off of the mouthpiece so as to form a roof of a tongue recess, and is configured so as not to interfere with the flow of the particles; b) fitting the mouthpiece to a subject in need of inhaling aerosolized particles wherein the subject places the apex of the subject's tongue into the tongue recess; and c) creating aerosolized particles with the nebulizer under conditions such that the particles flow into the inhalation airflow-path as the subject inhales, wherein the subject's tongue remains in the tongue recess as the subject inhales, thereby controlling the flow of the particles.

In another embodiment, the invention provides a method wherein the aerosol particles are created in synchrony with an inhalation phase of a breathing cycle of the subject. In still another embodiment, the emission is conditioned on a predetermined rate of breathing. In an exemplary embodiment, the rate of breathing is slow compared to tidal breathing. In another exemplary embodiment, the placement of the tongue in the tongue recess reduces the amount of particles deposited in the oral cavity and oropharynx, and increases the amount of particles deposited in the lungs of the subject. In still another embodiment, the mass median aerodynamic diameter of the particles is between 0.5 and 5 microns.

In yet another embodiment, the invention provides a kit comprising a device and instructions, the device comprising a nebulizer, a mouthpiece, and a tongue guide, wherein the mouthpiece comprises a breathing passage extending therethrough, wherein the breathing passage accommodates an inhalation airflow-path, wherein the nebulizer is disposed in proximity to the mouthpiece such that, when the nebulizer emits aerosol particles, the particles flow into the inhalation airflow-path as a subject inhales, wherein the tongue guide extends off of the mouthpiece so as to form a roof of a tongue recess, wherein the instructions instruct a subject to place the subject's tongue in the tongue recess before inhaling the particles. In one kit embodiment, the instructions further instruct a subject to inhale slowly while maintaining the tongue in the tongue recess.

In another embodiment, a drug delivery apparatus for delivering an aerosol including a drug to a patient is provided that includes an aerosol generator for generating the aerosol from a supply of the drug, and a mouthpiece component structured to be inserted into a mouth of the patient, the mouthpiece component being operatively coupled to the aerosol generator for receiving the aerosol and delivering the aerosol to the patient, the mouthpiece component having a ventral surface structured to face a tongue of the patient when the mouthpiece component is inserted into the mouth of the patient and a tongue apex positioning element provided on the ventral surface, wherein the tongue apex positioning element is structured to engage an apex of the tongue of the patient during use of the drug delivery apparatus.

In still another embodiment, a drug delivery apparatus for delivering an aerosol including a drug to a patient is provided that includes an aerosol generator for generating the aerosol from a supply of the drug, a mouthpiece component structured to be inserted into a mouth of the patient, the mouthpiece component being operatively coupled to the aerosol generator for receiving the aerosol and delivering the aerosol to the patient, the mouthpiece component having a ventral surface structured to face a tongue of the patient when the mouthpiece component is inserted into the mouth of the patient, and a controller controlling operation of the aerosol generator, wherein the controller is adapted to cause the drug delivery apparatus to provide instructions to the patient, the instructions instructing the patient to place an apex of the tongue against the ventral surface of the mouthpiece component during use of the drug delivery apparatus.

In still another embodiment, a kit for delivering an aerosol including a drug to a patient is provided that includes: (i) drug delivery apparatus including: an aerosol generator for generating the aerosol from a supply of the drug; and a mouthpiece component structured to be inserted into a mouth of the patient, the mouthpiece component being operatively coupled to the aerosol generator for receiving the aerosol and delivering the aerosol to the patient, the mouthpiece component having a ventral surface structured to face a tongue of the patient when the mouthpiece component is inserted into the mouth of the patient; and (ii) instructions instructing the patient to place an apex of the tongue against the ventral surface of the mouthpiece component during use of the drug delivery apparatus.

In still a further embodiment, a method of delivering an aerosol including a drug to a patient is provided that includes (i) providing a drug delivery apparatus including: an aerosol generator for generating the aerosol from a supply of the drug; and a mouthpiece component structured to be inserted into a mouth of the patient, the mouthpiece component being operatively coupled to the aerosol generator for receiving the aerosol and delivering the aerosol to the patient, the mouthpiece component having a ventral surface structured to face a tongue of the patient when the mouthpiece component is inserted into the mouth of the patient; (ii) instructing the patient to place an apex of the tongue against the ventral surface of the mouthpiece component during use of the drug delivery apparatus; and (iii) delivering the aerosol to the patient through the mouthpiece component when the apex of the tongue of the patient is placed against the ventral surface of the mouthpiece component.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

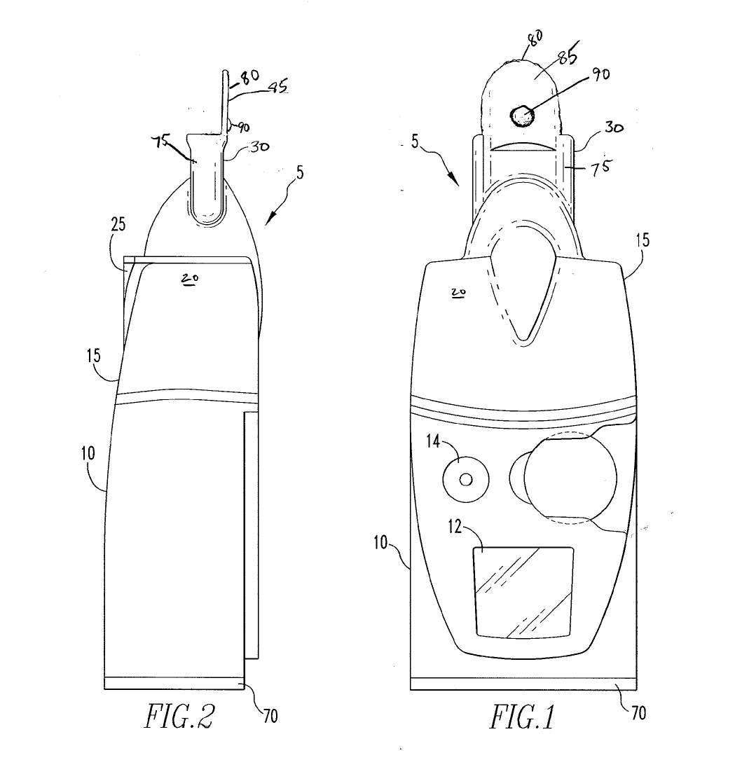

FIG. 1 is a front elevational view and FIG. 2 is a left side elevational view of a nebulizer device according to one, exemplary embodiment of the invention;

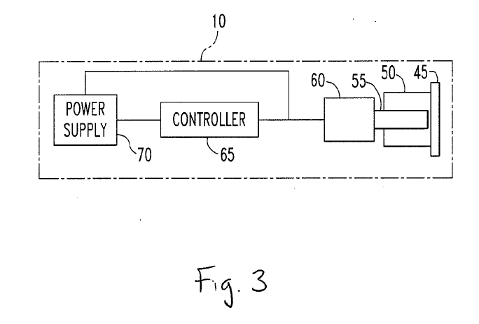

FIG. 3 is a schematic diagram of the nebulizer device of FIGS. 1 and 2 which shows selected components thereof in a simplified or symbolic form;

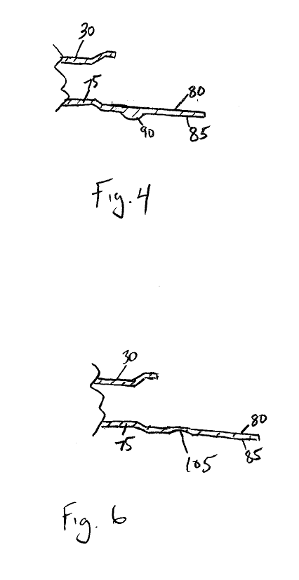

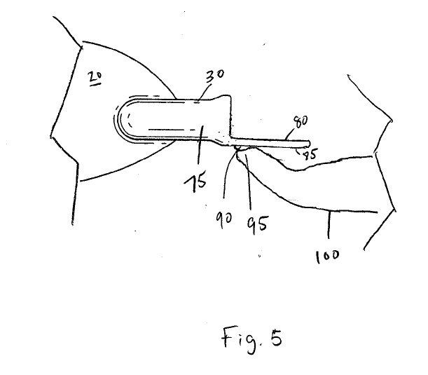

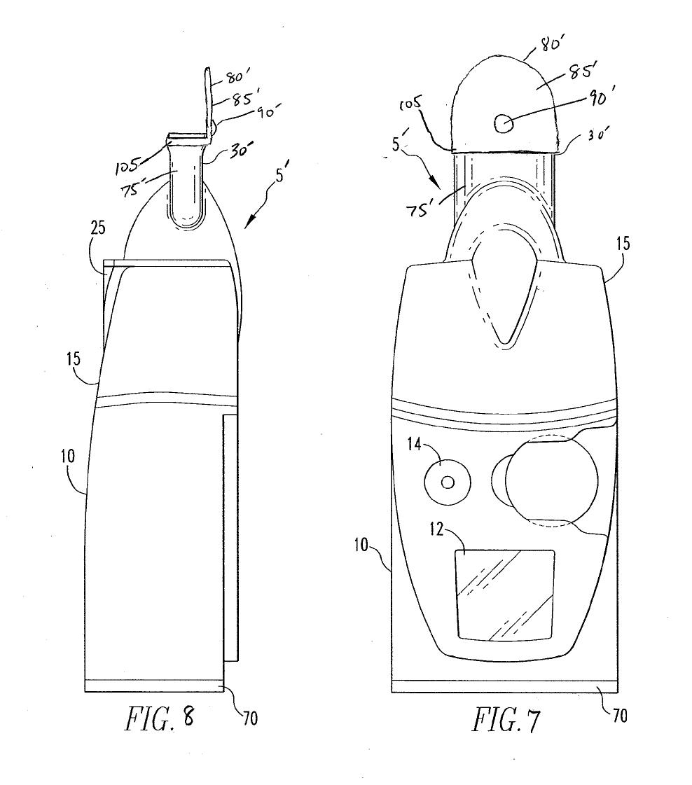

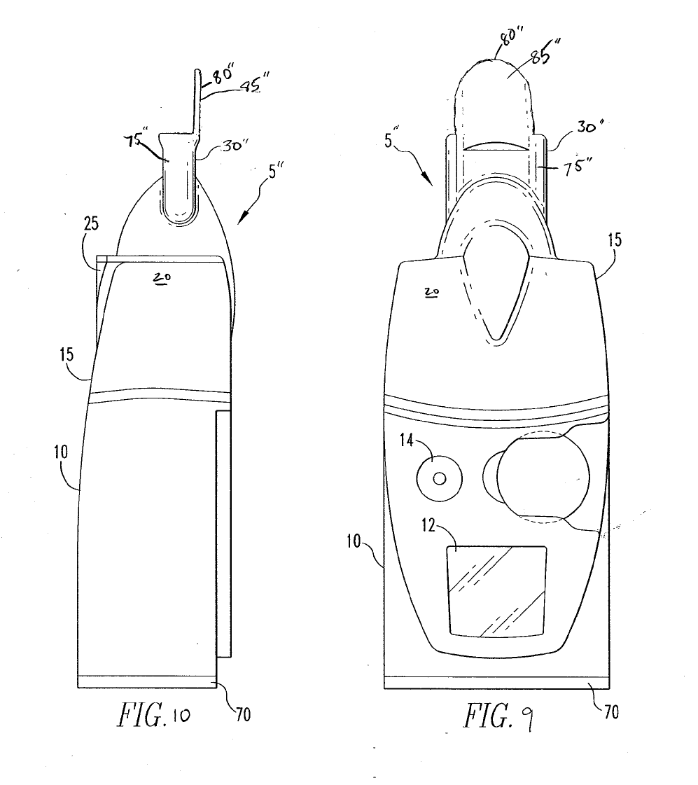

FIG. 4 is a cross-sectional view of the mouthpiece component forming a part of the nebulizer device of FIGS. 1 and 2;

FIG. 5 is a schematic diagram showing use of the mouthpiece component forming a part of the nebulizer device of FIGS. 1 and 2;

FIG. 6 is a cross-sectional view of an alternative mouthpiece component that may form a part of the nebulizer device of FIGS. 1 and 2;

FIG. 7 is a front elevational view and FIG. 8 is a left side elevational view of a nebulizer device according to an alternative, exemplary embodiment of the invention;

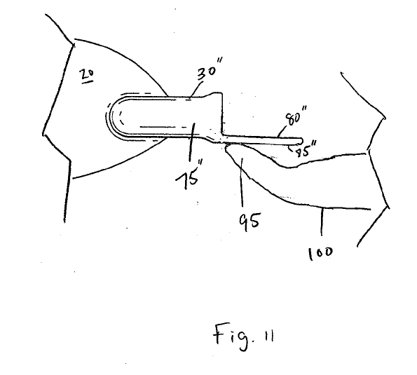

FIG. 9 is a front elevational view and FIG. 10 is a left side elevational view of a nebulizer device according to another alternative, exemplary embodiment of the invention;

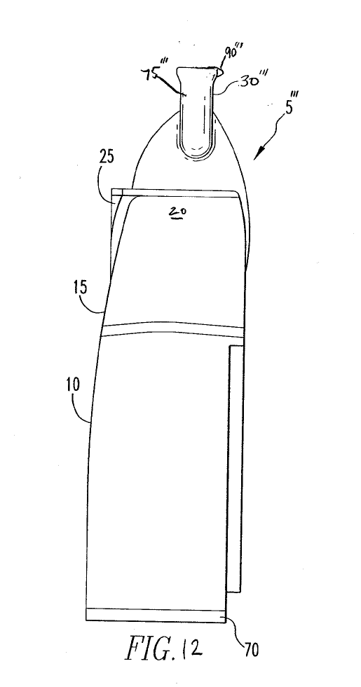

FIG. 11 is a schematic diagram showing use of the mouthpiece component forming a part of the nebulizer device of FIGS. 9 and 10; and

FIG. 12 is a left side elevational view of a nebulizer device according to a further alternative, exemplary embodiment of the invention.

To facilitate understanding of the descriptions herein of embodiments of the invention, a number of terms (set off in quotation marks) are defined below. Terms defined herein (unless otherwise specified) have meanings as commonly understood by a person of ordinary skill in the areas relevant to the present invention.

As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the invention, but their usage does not delimit the invention, except as outlined in the claims.

The phrase “chosen from A, B, and C” as used herein, means selecting one or more of A, B, C.

As used herein, absent an express indication to the contrary, the term “or” when used in the expression “A or B,” where A and B may refer to a composition, object, disease, product, etc., means one or the other (“exclusive OR”), or both (“inclusive OR”).

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the context dictates otherwise. For example a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities in the specification and claims are to the understood as being modified in all instances by the term “about.” Accordingly, unless indicated to the contrary, the numerical parameters in the specification and claims are approximations that may vary depending upon the desired properties sought to be obtained in a particular embodiment of the present invention. At the very least, and without limiting the application of the doctrine of equivalents to the scope of the claims, each numerical parameter should be construed in light of the number of reported significant digits and by applying ordinary rounding techniques. Any numerical value, however, inherently contains deviations that necessarily result from the errors found in the numerical value's testing measurements.

The term “not” when preceding, and made in reference to, any particularly named entity or phenomenon means that only the particularly named entity or phenomenon is excluded.

The term “altering” and grammatical equivalents as used herein in reference to any entity and/or phenomenon refers to an increase and/or decrease in the quantity of the entity in a given space and/or the intensity, force, energy or power of the phenomenon, regardless of whether determined objectively, and/or subjectively.

The terms “increase,” “elevate,” “raise,” and grammatical equivalents when used in reference to the quantity of an entity and/or the intensity, force, energy or power of a phenomenon in a first sample relative to a second sample, mean that the quantity of the entity and/or the intensity, force, energy or power of the phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, clarity of vision, etc. In another embodiment, the quantity of the substance and/or phenomenon in the first sample is at least 10% greater than the quantity of the same substance and/or phenomenon in the second sample. In another embodiment, the quantity of the substance and/or phenomenon in the first sample is at least 25% greater than the quantity of the same substance and/or phenomenon in the second sample. In yet another embodiment, the quantity of the substance and/or phenomenon in the first sample is at least 50% greater than the quantity of the same substance and/or phenomenon in the second sample. In a further embodiment, the quantity of the substance and/or phenomenon in a first sample is a least 75% greater than the quantity of the same substance and/or phenomenon in the second sample. In yet another embodiment, the quantity of the substance and/or phenomenon in the first sample is at least 90% greater than the quantity of the substance and/or phenomenon in the second sample. Alternatively, a difference may be expressed as an “n-fold” difference.

The terms “reduce,” “inhibit,” “diminish,” “suppress,” “decrease,” and grammatical equivalents when used in reference to the quantity of an entity and/or the intensity, force, energy or power of a phenomenon in a first sample relative to a second sample, mean that the quantity of the entity and/or the intensity, force, energy or power of the phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, weakness, etc. In another embodiment, the quantity of an entity and/or the intensity, force, energy or power of a phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in the second sample. Alternatively, a difference may be expressed as an “n-fold” difference.

The term “breathing passage” is used herein particularly in relation to the mouthpiece and refers to the opening in the mouthpiece through which the aerosol generator is in fluid communication with the oral cavity. The term “inhalation flow-path” or “inhalation airflow-path” refers to any passageway or combination of passageways along which inhaled air flows from a mouthpiece to the trachea. It is to be understood that an inhalation flow-path need not remain constant in dimension or distribution throughout an inhalation, from inhalation to inhalation, between subjects, or between administrations of aerosol.

The term “mouthpiece” refers herein to any device that provides the aforementioned opening. Mouthpieces most advantageously employed in embodiments of the present invention, however, do not have simply a tongue depressor (or deflector) as an element. As used herein, “tongue depressors” and “tongue deflectors” refer to devices that depress the tongue or a portion thereof toward the floor of the mouth or deflect it to the side of the mouth without the direct cooperation or action of the subject. A variety of tongue depressor configurations have been incorporated into mouthpieces to provide this function. U.S. Pat. Nos. 4,148,308, 5,533,523, 6,606,992, 7,140,365 and 7,464,706, and U.S. Patent Application Publication No. 2007/0221211 are exemplary. Although the subject, in each of these examples, is obliged to self-insert the tongue depressor, the subject is passive in the tongue depression itself. The subject, in other words, does not perform any active steps with his or her tongue to get his or her tongue out of the way. Instead, the force of the tongue depressor get his or her tongue out of the way.

The term “tongue guide” refers generally herein to any means that (1) assists a subject to position his or her tongue so that its dorsal surface tends not to contact the roof of the subject's mouth when the subject is inhaling aerosolized particles, and (2) preferably covers the dorsal surface of the tongue posteriorly by less than about 3 cm from the tip or apex, more preferably by less than 2 cm and most preferably by less than 1 cm. In fact, the guide may be merely or include a tongue apex positioning element, such as, without limitation a prominence, such as a bump, or a depression disposed on a ventral aspect of a part of the mouthpiece that, in use, resides in the mouth. The bump need only be sufficiently large to be sensible by the apex or tip of the tongue of a conscious subject. Preferably, however, it is large enough to form the roof of a tongue recess for the anterior aspect of the tongue, which recess may be further defined by the floor of the mouth and the lower gums or the lingual surface of the lower teeth interiorly. It is not necessary that the roof portion provide a patent cover for the tongue. That is, the roof may be fenestrated in any of a variety of ways. The objective of the tongue recess is to assist the subject to position the tongue, not to protect it.

The term “interfere with,” as used herein relates especially to the flow of air or of aerosolized particles, and makes no assumptions as to the mechanism of interference. An ideal flow condition would be laminar flow at a constant (preferably slow) rate from mouthpiece to trachea, without turbulence. It will be understood that although such an ideal condition is not likely to be attained in practice, any circumstance that biases toward this ideal is desirable.

As used herein, the terms “inhalation” and “inspiration” (used interchangeably) refer to an intake of air into a lung, generally impelled along a pressure gradient wherein a negative pressure is established by an expansion of a subject's pleural cavity, the expansion effected mainly by a contraction of the subject's diaphragm. It is not intended, however, to limit the applicability of the terms of any particular mechanism. Inspiration may be accomplished by positive instead of negative pressure or by an inspiration effected by an iron lung, for example. In general, inspiration occurs in the context of a breathing cycle or respiratory cycle wherein expiration or exhalation alternates with inspiration, generally at a relatively constant “breathing rate” depending upon physiological demands on the subject, voluntary adjustments by the subject and anatomical or other constraints imposed upon the subject.

The term “wearing” as used herein in relation to mouthpieces refers to the mouthpiece in use (i.e., “fitted” to a subject) and is intended to encompass mouthpieces ranging in complexity from simple tubular conduits to mouthpieces fitted with collars, bite-mounts, head-straps, etc.

“Nebulizers” refer herein to any device that is capable of generating aerosols, such as, without limitation, nebulizers that produce liquid droplets (“wet” aerosols) and nebulizers that provide energy to generate their aerosol. In general, the aerosol generated by a nebulizer appears as a “cloud” or “mist,” often called a “plume” that tends to drift at low velocity (unless it is injected by a faster moving stream of air). An exemplary but non-limiting nebulizer that may be used in embodiments of the present invention is the i-Neb® AAD® system sold by Philips Respironics of Murrysville, Pa.

The term “proximity” as used herein refers particularly to the relationship between a nebulizer and a mouthpiece. Preferably, nebulizer and mouthpiece will be attachable or actually attached to one another. The term, however, extends to nebulizers that are near enough to a mouthpiece to permit the nebulizer's plume to reach or be inhaled into the mouthpiece.

The term “synchronized” is used herein in relation to the breath-actuation of nebulizers. Typically, breath-actuated nebulizers automatically release an aerosol plume when (or shortly before) the subject begins to inhale. Herein, the term is not limited to “automatic” breath-actuation. For example, actuation of the nebulizer might be under the control of a subject who actuates the device in response to a prompt, which prompt occurs “in synchrony” with the occurrence of a particular event or combination of events. For example, the prompt may occur at the moment of an expected inhalation after a subject's monitored breathing rate stabilizes to a predetermined level.

As employed, herein, the statement that two or more parts or components are “coupled” together shall mean that the parts are joined or operate together either directly or through one or more intermediate parts or components.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As described elsewhere herein, an increasing number of expensive drugs are being administered to the lungs in aerosol form. Interferon-γ, for example, has been shown to be effective in treating idiopathic pulmonary fibrosis when administered directly to the lungs in an aerosol (U.S. Pat. No. 6,964,761). Similar evidence for asthma (U.S. Patent Application Publication No. 2007/0065367) and tuberculosis (U.S. Patent Application Publication No. 2008/0292559) exists. The costliness of drugs such as interferon, and the potential for adverse effects when dosages deviate from the expected, has focused a great deal of attention on finding optimum conditions for aerosol therapy. At the same time, the benefits of such drugs are not limited to diseases so ominous that the patient is willing to undergo significant discomfort to effect the treatment. Thus, it is desirable to avoid extreme or unpleasant measures such as endotracheal intubation, or even tongue depressors or other devices, e.g., metal or plastic conduits to avoid or eliminate oropharyngeal tissues as deposition sites. It is also advantageous to avoid devices that compromise or complicate the flow of inhaled air.

The behavior of particles suspended in a given volume of flowing air depends strongly upon the rate at which the air is flowing. Aerosols are colloids, which is to say that the particles comprising them form stable suspensions in air. Thus, even quite large (heavy) particles in an aerosol, being attracted to one another to an extent, tend to “float” or “slide” over or around surfaces if the air in which they are borne is flowing slowly enough. On the other hand, the momentum of even small (light) particles suspended in a fast-moving volume of air may be enough to cause the particles to impact surfaces with sufficient force to make them stick.

It is an object of the present invention to provide a capacious path for air flowing from the mouthpiece of a nebulizer or similar drug delivery apparatus to the trachea of the user. That is, a pathway relatively unobstructed by the tongue is to be preferred. Others have called attention to this preference. Svartengran et. al (Eur. Respir. J. 9: 1556-1559, 1996), for example, noted that >70% of inhaled aerosol is deposited in the oropharynx in some patients. The authors attempted to ameliorate the effect by using a mouthpiece elongated sufficiently to bypass the oral cavity and the tongue. It was not possible, however, because of gagging and general discomfort, to use a mouthpiece that extended posteriorly beyond the hard palate. Although the elongated mouthpiece provided some reduction in oropharyngeal deposition, the difference was not significant.

More effective than this attempt to reduce deposition by using a conduit to by-pass the tongue have been efforts to control the rate of inhalation and the size of administered aerosolized particles. U.S. Patent Application Publication No. 2008/0292559, for example, discloses that a pattern of breathing using a method of slow and deep inspiration as compared to tidal breathing enforces a reduction in inspiratory flow-rate and a greatly prolonged inspiratory time. The slow inspiration allows aerosol particles to bypass the upper airways thus making them available for deposition in the lung.

The size of administered particles appears to be an optimization problem. In general, smaller (lighter) particles are better suited than larger particles to remain in colloidal suspension during close encounters with oropharyngeal surfaces. However, prolonged inspiration allows for suitable settling of completely inhaled aerosols in the lung periphery. The prolongation of the inspiratory time and the advanced settling promotes “inspiratory deposition” before the inhaled particles can be exhaled. As noted in U.S. Patent Application Publication No. 2008/0292559, it is possible under these circumstances to have almost 100% of the inhaled particles depositing before exhalation begins. This process can be further enhanced by using particles that are relatively large (e.g., about 4.5μ) and would ordinarily deposit in the oropharynx. The prolonged inspiration of slow and deep breathing is particularly suited for delivery of drugs to the lungs of patients whose peripheral airway pathology results in reduced deposition of conventional smaller aerosols as well as promoting avoidance of deposition in the oropharynx. Diseases of the lung periphery that may be treated by this method include, for example, idiopathic pulmonary fibrosis and emphysema. Both these entities result in enlarged airspaces within the lung that result in minimal deposition during tidal breathing.

In addition to conjointly optimizing the rate of airflow and the size of aerosol particles, a means for establishing and maintaining a wide flow-path during inhalation is advantageous. A means that is innocuous to the user is even more advantageous. The present inventor has found, in connection with treating patients for idiopathic pulmonary fibrosis, that intubation, long mouthpieces that activate the gag reflex, and even tongue depressors are unnecessary. Subjects can readily orient their tongues so as to create an inhalation flow-path of substantial capacity. All that is needed is a “cue” that guides a conscious subject's tongue to assume such a position. The applicant has found that adding an extension, for example using a targeting element, to the ventral surface of the mouthpiece distally provides an adequate cue.

As stated elsewhere herein, the i-Neb® AAD® system sold by Philips Respironics of Murrysville, Pa. is a suitable nebulizer that may be used in connection with the present invention. It is to be noted that the i-Neb® AAD® system is breath-actuated, subject to a measure of control by the subject. That is, the aerosol delivery system is adaptive. The aerosol is created through vibrating mesh technology, and the dosage of drug is controlled through electronic feedback and specific metering chambers. The metering chambers can deliver a pre-set volume ranging from 0.25 to 1.4 mL with a residual of about 0.1 mL. The vibrating mesh has a variable power range for the optimization of the aerosol output. In addition, the i-Neb® AAD® system incorporates an algorithm that pulses medication delivery into 50 to 80 percent of each inspiration, based on a rolling average of the last three breaths. Throughout the treatment, the i-Neb® AAD® system provides continuous feedback to the patient through a liquid crystal display, and upon successful delivery of the treatment, the patient receives audible and tactile feedback. The subject is thus provided with sufficient real-time information to operate the system optimally with respect to breathing rate.

In studies conducted by the present inventor wherein treatments were provided both with the existing i-Neb® AAD® system and a i-Neb® AAD® system modified to include an extension to the ventral surface of the mouthpiece distally, it has been established that particle size as determined by mass median aerodynamic diameter (MMAD) did not vary between treatments. The main distinction between the two treatments was the percentage of particles (measured as radioactivity) that accumulated in the oropharyngeal cavity. The subject “recovered” particles deposited in the oral cavity by drinking water at the end of treatment. Thus the radioactivity in the stomach approximately equals the radioactivity deposited in the oral cavity. It is concluded that controlling the position of the tongue to make a clear inhalation flow-path is within the capability of the subject, that such control is beneficial to treatment, and that the modified mouthpiece, which is the subject of the present invention, effectively assists the subject in this respect. Sample data from the studies is provided in Table 1 below.

TABLE 1

| | Original Mouthpiece | Modified Mouthpiece |
|---|---|---|
| Study date | Mar. 12, 2009 | Apr. 24, 2009 |
| Mesh ID | 90 | 90 |
| MMAD | 2.0 | 2.0 |
| aC/P | 0.78 | 0.80 |
| sC/P | 1.10 | 1.14 |
| % Lung Deposition | 25.2 | 39.8 |
| % Stomach Deposition | 45.9 | 7.1 |
| % Exhaled Activity | 0.0 | 5.9 |
| % Drug Residual | 31.8 | 31.3 |
| Tx Run Time (min) | 4.43 | 6.43 |

As previously noted, although the i-Neb® AAD® system was advantageous herein for helping to control breathing rate and constancy of dose, the choice of nebulizer is not limiting, and the present invention may be used in connection with any nebulizer or similar drug delivery apparatus.

The use of the adapted mouthpiece is straightforward. The subject inserts the modified mouthpiece of the present invention exactly as if it were an original but seeks out the extension, for example using a targeting element, coupled to the ventral surface of the mouthpiece distally with the tip of the tongue and places the tip of the tongue in contact therewith preferably for the duration of the treatment but at least prior to and during each inhalation. It is also possible to provide the modified mouthpiece and/or an adapter comprising an extension for attachment to an original mouthpiece, with or without a nebulizer, as a kit with instructions for use.

FIG. 1 is a front elevational view and FIG. 2 is a left side elevational view of nebulizer device 5 according to one particular exemplary embodiment of the invention. Nebulizer device 5 functions as a drug delivery system for delivering a drug in the form of an aerosol into the lungs of a patient. Nebulizer device 5 includes main housing 10, which houses certain components of nebulizer device 5, and mouthpiece portion 15, which is removeably attached to main housing 10. FIG. 3 is a schematic diagram of main housing 10 of nebulizer device 5 which shows selected components thereof (described below) in a simplified or symbolic form.

Mouthpiece portion 15 includes main chamber 20 which, when mouthpiece portion 15 is attached to the main housing 10, is structured to receive the aerosol that is generated by the components in main housing 10 as described in more detail below. Main chamber 20 includes air inlet 25. In addition, main chamber 20 has an internal conduit leading to mouthpiece component 30 that is structured to be received in the mouth of the patient. When the patient places his or her mouth on mouthpiece component 30 and inhales, air is caused to flow into chamber 20 from air inlet 25 and through the internal conduit of main chamber 20 to mouthpiece component 30. As will be appreciated, that air stream carries the aerosol that is generated in the manner described below into the lungs of the patient.

As seen in FIG. 3, main housing 10 includes mesh plate 45 (including a plurality of miniature holes therein), reservoir 50 for holding the liquid (drug) to be converted into an aerosol, horn 55, and piezoelectric transducer 60 operatively coupled to horn 55. Main housing 10 also includes controller 65 and power supply 70, which preferably is a rechargeable battery. Controller 65 includes a processing portion which may be, for example, a microprocessor, a microcontroller or some other suitable processing device, and a memory portion that may internal to the processing portion or operatively coupled to the processing portion and that provides a storage medium for data and software executable by the processing portion for controlling the operation of nebulizer 5, including providing the feedback and instructions as described in greater detail herein.

Horn 55 is located close to the rear face of mesh plate 45 and may be caused to vibrate by piezoelectric transducer 60 under the control of controller 65, with the power to drive the piezoelectric transducer 60 being provided by power supply 70. The liquid in reservoir 50 is in fluid contact with the rear face of mesh plate 45. When piezoelectric transducer 60 is caused to vibrate, it drives horn 55 to vibrate in the region of mesh plate 45. As a result of such vibration of horn 55, the liquid from reservoir 50 is forced through the holes of mesh plate 45, thereby generating an aerosol plume that is injected into main chamber 20. As seen in FIG. 1, main housing 10 includes LCD 12 for providing information to the patient about the treatment and operation of nebulizer device 5, and button 14 for providing input for controlling various aspects of the nebulizer device 5.

It should be understood that the mesh plate type aerosol generation system shown in FIG. 3 and just described is meant to be exemplary and is just one type of aerosol generation system that may be employed in connection with the present invention. It is contemplated that others type of aerosol generation systems may be employed within the scope of the present invention. For example, and without limitation, an aerosol generation system that employs a piezeo element around the outside of the mesh and not a separate horn as in FIG. 3 may be employed. As another non-limiting example, a pneumatic type aerosol generation system may be employed.

As seen in FIGS. 1 and 2, mouthpiece component 30 includes base portion 75 that includes top, bottom (ventral), left and right side walls for defining a breathing passage therein. Base portion 75 is fluidly coupled to main chamber 20 and thus is structured to receive the aerosol carrying air stream described above from main chamber 20. Mouthpiece component 30 also includes tongue guide 80 that is coupled to and extends outwardly from the bottom (ventral) wall of base portion 75. In addition, bottom surface 85 of tongue guide 80 is provided with a tongue apex positioning element, which in the illustrated embodiment is in the form of dome-shaped protruding member 90 extending from bottom surface 85. FIG. 4 is a cross-sectional view of mouthpiece component 30 which shows protruding member 90.

In one particular, non-limiting embodiment, nebulizer device has the following dimensions: width of main housing 10: 65 mm; depth of main housing 10: 45 mm; distance from bottom of main housing 10 to distal end of base portion 75: 151 mm; distance from bottom of mouthpiece portion 15 to distal end of base portion 75: 76 mm; width of base portion 75: 25.4 mm; and length of tongue guide 80: 19 mm.

Referring to FIG. 5, in use, a patient inserts mouthpiece component 30 into his or her mouth. Before inhaling aerosolized particles generated by nebulizer 5 as described herein, the patient places apex 95 of his or her tongue 100 against protruding member 90. This causes tongue 100 to be positioned in a manner that does not interfere with the flow of air or aerosol along the inhalation airflow-path extending from mouthpiece component 30 to the patient's trachea. Thus, protruding member 90 is, in the exemplary embodiment, sufficiently prominent such that it will be able to be readily sensed by apex 95 of tongue 100.

In one particular embodiment, controller 65 is adapted (programmed) to cause instructions to be provided to the user instructing the user to place apex 95 of his or her tongue 100 against protruding member 90 prior to and/or during treatment (for example, in response to the patient pressing button 14 to commence aerosol generation). Such instructions may be provided, for example and without limitation, visually, through LCD 12, audibly, through a speaker provided as part of nebulizer 5, or both visually and audibly.

It should be appreciated that the embodiment shown in FIGS. 1, 2, 4 and 5 is meant to be exemplary only, and that the tongue apex positioning element provided on bottom surface 85 of tongue guide 80 may have other forms. For example, referring to FIG. 6, the tongue apex positioning element may comprise a depression or recessed portion 105, such as an elongated groove or an inverted dome-shaped recess, provided in bottom surface 85 of tongue guide 80. In this embodiment, the patient will, when using nebulizer 5 (and possibly in response to instructions as described above), seek out depression 105 with apex 95 (e.g., apex 95 may be received in depression 105) in order to position tongue 100 as described above.

A further alternative embodiment is shown in FIGS. 7 and 8. In that embodiment, nebulizer 5′ is similar to nebulizer 5 shown in FIGS. 1 and 2, and like components are labeled with like reference numerals. Nebulizer 5′, however, includes mouthpiece component 30′ that comprises base portion 75′ and selectively attachable tongue guide 80′. In particular, selectively attachable tongue guide 80′ includes bottom surface 85′ having a tongue apex positioning element, which in the illustrated embodiment is in the form of dome-shaped protruding member 90′. However, rather than being permanently attached to attached base portion 75′, tongue guide 80′ includes ring portion 105 that has a shape that is similar to the shape of the distal end of base portion 75′ to enable tongue guide 80′ to be attached to base portion 75′ by way of a snap fit, a friction fit, or some other suitable method. As such, tongue guide 80′ may be used with existing nebulizers that were not originally provided with such a device.

A still further alternative embodiment is shown in FIGS. 9 and 10. In that embodiment, nebulizer 5″ is similar to nebulizer 5 shown in FIGS. 1 and 2, and like components are labeled with like reference numerals. Nebulizer 5″, however, includes mouthpiece component 30″ that comprises base portion 75″ coupled to tongue guide 80″ that does not include a tongue apex positioning element. In this embodiment, in use, the patient places apex 95 of his or her tongue 100 against bottom surface 85″ of tongue guide 80″ as shown in FIG. 11. In one particular embodiment, controller 65 is adapted (programmed) to cause instructions to be provided to the user instructing the user to place apex 95 of his or her tongue 100 against bottom surface 85″ of tongue guide 80″ prior to and/or during treatment (for example, in response to the patient pressing button 14 to commence aerosol generation). Such instructions may be provided, for example and without limitation, visually, through LCD 12, audibly, through a speaker provided as part of nebulizer 5, or both visually and audibly.

Yet a further alternative embodiment is shown in FIG. 12. In that embodiment, nebulizer 5′″ is similar to nebulizer 5 shown in FIGS. 1 and 2, and like components are labeled with like reference numerals. Nebulizer 5′″, however, includes mouthpiece component 30′″ that comprises base portion 75′″ having a tongue apex positioning element in the form of protruding member 90′″ provided on the bottom wall of base portion 75′″. In this embodiment, in use, the patient places apex 95 of his or her tongue 100 against protruding member 90′″. In one particular embodiment, controller 65 is adapted (programmed) to cause instructions to be provided to the user instructing the user to place apex 95 of his or her tongue 100 against protruding member 90′″ prior to and/or during treatment (for example, in response to the patient pressing button 14 to commence aerosol generation). Such instructions may be provided, for example and without limitation, visually, through LCD 12, audibly, through a speaker provided as part of nebulizer 5, or both visually and audibly.

Referring again to FIGS. 7 and 8, an alternative selectively attachable tongue guide 80′ that does not include a tongue apex positioning element may also be provided, which functions as just described in connection with tongue guide 80″.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A drug delivery apparatus (5, 5 •, 5 • • •) for delivering an aerosol including a drug to a patient, comprising:
an aerosol generator (45, 50, 55, 60) for generating the aerosol from a supply of the drug; and
a mouthpiece component (30, 30 •, 30 • • •) structured to be inserted into a mouth of the patient, the mouthpiece component being operatively coupled to the aerosol generator for receiving the aerosol and delivering the aerosol to the patient, the mouthpiece component having a ventral surface (85, 85 •) structured to face a tongue (100) of the patient when the mouthpiece component is inserted into the mouth of the patient and a tongue apex positioning element (90, 90 •, 90 • • •, 105) provided on the ventral surface, wherein the tongue apex positioning element is structured to engage an apex (95) of the tongue of the patient during use of the drug delivery apparatus.

2. The drug delivery apparatus according to claim 1, wherein the mouthpiece component comprises a base portion (75, 75 •, 75 • •) and a tongue guide (80, 80 •, 80 • •) extending from a bottom wall of the base portion, wherein the ventral surface is a bottom surface of the tongue guide.

3. The drug delivery apparatus according to claim 2, wherein the tongue guide is integral with the base portion.

4. The drug delivery apparatus according to claim 2, wherein the tongue guide is selectively attachable to a distal end of the base portion.

5. The drug delivery apparatus according to claim 2, wherein the tongue apex positioning element comprises a protruding member (90, 90 •) protruding from the ventral surface.

6. The drug delivery apparatus according to claim 5, wherein the protruding member is dome-shaped (90, 90 •).

7. The drug delivery apparatus according to claim 2, wherein the tongue apex positioning element comprises a recess (105) provided in the ventral surface.

8. The drug delivery apparatus according to claim 1, further comprising a controller (65) controlling operation of the aerosol generator, wherein the controller is adapted to cause the drug delivery apparatus to provide instructions to the patient, the instructions instructing the patient to place the apex of the tongue against the tongue apex positioning element during use of the drug delivery apparatus.

9. The drug delivery apparatus according to claim 8, wherein the instructions comprise at least one of visual instructions provided on a display (12) of the drug delivery apparatus and audible instructions.

10. A drug delivery apparatus (5, 5 •, 5 • •, 5 • • •) for delivering an aerosol including a drug to a patient, comprising:
an aerosol generator (45, 50, 55, 60) for generating the aerosol from a supply of the drug;
a mouthpiece component (30, 30 •, 30 • •, 30 • • •) structured to be inserted into a mouth of the patient, the mouthpiece component being operatively coupled to the aerosol generator for receiving the aerosol and delivering the aerosol to the patient, the mouthpiece component having a ventral surface (85, 85 •, 85 • •) structured to face a tongue (100) of the patient when the mouthpiece component is inserted into the mouth of the patient; and
a controller (65) controlling operation of the aerosol generator, wherein the controller is adapted to cause the drug delivery apparatus to provide instructions to the patient, the instructions instructing the patient to place an apex (95) of the tongue against the ventral surface of the mouthpiece component during use of the drug delivery apparatus.

11. The drug delivery apparatus according to claim 10, wherein the mouthpiece component comprises a base portion (75, 75 •, 75 • •) and a tongue guide (80, 80 •, 80 • •) extending from a bottom wall of the base portion, wherein the ventral surface is a bottom surface of the tongue guide.

12. The drug delivery apparatus according to claim 11, wherein the tongue guide is integral with the base portion.

13. The drug delivery apparatus according to claim 11, wherein the tongue guide is selectively attachable to a distal end of the base portion.

14. The drug delivery apparatus according to claim 10, wherein the instructions comprise at least one of visual instructions provided on a display of the drug delivery apparatus and audible instructions.

15. A kit for delivering an aerosol including a drug to a patient, comprising:
(i) drug delivery apparatus (5, 5 •, 5 ••, 5 • • •) including:
an aerosol generator (45, 50, 55, 60) for generating the aerosol from a supply of the drug; and
a mouthpiece component (30, 30 •, 30 • •, 30 • • •) structured to be inserted into a mouth of the patient, the mouthpiece component being operatively coupled to the aerosol generator for receiving the aerosol and delivering the aerosol to the patient, the mouthpiece component having a ventral surface (85, 85 •, 85 • •) structured to face a tongue (100) of the patient when the mouthpiece component is inserted into the mouth of the patient; and
(ii) instructions instructing the patient to place an apex (95) of the tongue against the ventral surface of the mouthpiece component during use of the drug delivery apparatus.

16. The kit according to claim 15, wherein the mouthpiece component comprises a base portion (75, 75 •, 75 • •) and a tongue guide (80, 80 •, 80 • •) extending from a bottom wall of the base portion, wherein the ventral surface is a bottom surface of the tongue guide.

17. The kit according to claim 16, wherein the tongue guide is integral with the base portion.

18. The kit according to claim 16, wherein the tongue guide is selectively attachable to a distal end of the base portion.

19. The kit according to claim 15, wherein the ventral surface includes a tongue apex positioning element (90, 90 •, 90 • • •, 105), wherein the tongue apex positioning element is structured to engage an apex of the tongue of the patient during use of the drug delivery apparatus, wherein the instructions instruct the patient to place the apex of the tongue against the tongue apex positioning element during use of the drug delivery apparatus.

20. The kit according to claim 19, wherein the tongue apex positioning element comprises a protruding member (90, 90 •, 90 • • •) protruding from the ventral surface.

21. The kit according to claim 19, wherein the tongue apex positioning element comprises a recess (105) provided in the ventral surface.

22. A method of delivering an aerosol including a drug to a patient, comprising:
(i) providing a drug delivery apparatus (5, 5 •, 5 • •, 5 • • •) including:
an aerosol generator (45, 50, 55, 60) for generating the aerosol from a supply of the drug; and
a mouthpiece component (30, 30 •, 30 • •, 30 • • •) structured to be inserted into a mouth of the patient, the mouthpiece component being operatively coupled to the aerosol generator for receiving the aerosol and delivering the aerosol to the patient, the mouthpiece component having a ventral surface (85, 85 •, 85 • •) structured to face a tongue (100) of the patient when the mouthpiece component is inserted into the mouth of the patient;
(ii) instructing the patient to place an apex (95) of the tongue against the ventral surface of the mouthpiece component during use of the drug delivery apparatus; and
(iii) delivering the aerosol to the patient through the mouthpiece component when the apex of the tongue of the patient is placed against the ventral surface of the mouthpiece component.

23. The method according to claim 22, wherein the mouthpiece component comprises a base portion (75, 75 •, 75 • •) and a tongue guide (80, 80 •, 80 • •) extending from a bottom wall of the base portion, wherein the ventral surface is a bottom surface of the tongue guide.

24. The method according to claim 23, wherein the tongue guide is integral with the base portion.

25. The method according to claim 23, wherein the tongue guide is selectively attachable to a distal end of the base portion.

26. The method according to claim 15, wherein the ventral surface includes a tongue apex positioning element (90, 90 •, 90 • • •, 105), wherein the tongue apex positioning element is structured to engage an apex of the tongue of the patient during use of the drug delivery apparatus, wherein the instructing comprises instructing the patient to place the apex of the tongue against the tongue apex positioning element during use of the drug delivery apparatus.

27. The method according to claim 26, wherein the tongue apex positioning element comprises a protruding member (90, 90 •, 90 • • •) protruding from the ventral surface.

28. The method according to claim 26, wherein the tongue apex positioning element comprises a recess (105) provided in the ventral surface.
